(19) United States
(12) Patent Application Publication
(10) Pub. No.: US 2026/0142020 A1
(43) Pub. Date: May 21, 2026

(54) TRANSFER OF MEDICAL DATA FROM AN IMAGING SYSTEM TO A FILE TRANSFER LOCATION IDENTIFIED IN A PROFILE MAPPED TO IN A SCAN PROTOCOL

(71) Applicant: GE Precision Healthcare LLC, Waukesha, WI (US)

(72) Inventors: Chelsey Amanda Lewis, Brookfield, WI (US); Bradley Jay Gabrielse, Brookfield, WI (US); Margaret Carol Anderson, Milwaukee, WI (US); Matthew Flejter, Milwaukee, WI (US); Grant Alexander Brooks, Milwaukee, WI (US)

(73) Assignee: GE Precision Healthcare LLC, Waukesha, WI (US)

(21) Appl. No.: 18/952,844

(22) Filed: Nov. 19, 2024

(51) Int. Cl. G16H 30/20

(52) U.S. Cl. CPC G16H 30/20

(57) ABSTRACT

A medical imaging system includes a data acquisition system for acquiring projection data of a subject during a scan of the subject based on a scan protocol. The scan protocol includes a reconstruction with a mapping to a file transfer profile. The transfer profile includes a file transfer location to a host. The reconstruction does not include the file transfer location. The medical imaging system further includes a reconstructor configured to generate image data based on the acquired data and the scan protocol. The medical imaging system further includes an operator console with a processor configured to execute instructions that initiate transfer of the image data to the host based on the file transfer location in the file transfer profile mapped to in the reconstruction of the scan protocol.

### FIELD

The following generally relates to medical imaging and more particularly to transfer of medical data (e.g., image data, reports, etc.) from an imaging system to a file transfer location identified in a file transfer profile mapped to in a scan protocol.

### BACKGROUND

A Computed Tomography (CT) imaging system includes a gantry with a bore and a rotating frame rotatably supported in the gantry. The rotating frame includes an annular ring and is configured to rotate around the bore along an axis of rotation about a center of the bore. The rotating frame carries components such as an X-ray source and a detector array, which are disposed along arcs of the ring, opposite each other, across the bore. The rotating frame rotates around a subject, the X-ray source emits X-ray radiation that traverses the subject, and the detector array detects X-ray radiation traversing the subject and impinging the detector array.

The detector array generates projection data (line integrals) indicative of the detected X-ray radiation. A reconstructor reconstructs the projection data and generates volumetric image data. Voxels of the volumetric image data are displayed as a two-dimensional (2-D) and/or a three-dimensional (3-D) CT image using gray scale values corresponding to a relative radiodensity. The gray scale values reflect the attenuation characteristics of the scanned subject and show structure such as anatomical structures within the scanned subject. The CT images may also include colorized portions or overlays.

Prior to scanning a subject, a clinician sets up the scan for the subject. For this, in one instance, the clinician opens a scan session, enters patient identification information, and enters information for the scan into a scan protocol. In general, the scan protocol includes parameters such as acquisition parameters (e.g., X-ray tube voltage, X-ray tube current, slice thickness, scan range, rotation time, subject positioning, type of scan(s) (e.g., pre-scan/scout, helical and/or axial), etc.), reconstruction parameters (e.g., reconstructions algorithms, etc.), and post-imaging parameters (e.g., one or more file transfer locations, etc.)

With respect to the one or more file transfer locations, for each reconstruction of a scan protocol, the user manually enters and/or selects one or more network addresses such as Internet Protocol (IP) addresses to a host such as a Picture Archiving and Communication System (PACS), a backup server, a workstation with software with particular image processing features, etc. The imaging system automatically transfers the image data using the one or more file transfer locations. Where an imaging entity includes multiple imaging systems, the user has to configure each imaging system as such.

When a file transfer location changes, each reconstruction of each protocol on each imaging system that is affected by the change has to be updated. An example of a change that would affect a protocol is a newly added host that should receive the image data. Another example of a change that would affect a protocol is a change in a network address of a host currently receiving the image data. Another example of a change that would affect a protocol is the removal of a host where the network address is no longer used or is now used with a different host that is not intended to receive the image data.

In such instances, authorized personnel are burdened with manually updating each reconstruction of each protocol for each imaging system that is affected by the change. Unfortunately, this task consumes time that could otherwise be utilized in patient care, and utilizing an imaging system without updating the protocol would result in a loss of certain functionality such as no longer being able to automatically transfer image data to a host because the file transfer location in the protocol is no longer valid for the host.

In view of at least the foregoing, there is an unresolved need for an improved approach for configuring an imaging system for transferring data.

### SUMMARY

Aspects described herein address the above-referenced problems and others. This summary introduces concepts that are described in more detail in the detailed description. It should not be used to identify essential features of the claimed subject matter, nor to limit the scope of the claimed subject matter.

In one aspect, a medical imaging system includes a data acquisition system for acquiring projection data of a subject during a scan of the subject based on a scan protocol. The scan protocol includes a reconstruction with a mapping to a file transfer profile. The transfer profile includes a file transfer location to a host. The reconstruction does not include the file transfer location. The medical imaging system further includes a reconstructor configured to generate image data based on the acquired data and the scan protocol. The medical imaging system further includes an operator console with a processor configured to execute instructions that initiate transfer of the image data to the host based on the file transfer location in the file transfer profile mapped to in the reconstruction of the scan protocol.

In another aspect, a computer-implemented method includes receiving image data generated based on data acquired for a subject during a scan of the subject and a scan protocol. The scan protocol includes a mapping to a file transfer profile. The file transfer profile includes a file transfer location to a host. The scan protocol does not include the file transfer location to the host. The computer-implemented method further includes transferring the image data to the host based on the file transfer location in the file transfer profile.

In another aspect, a computer readable medium is encoded with computer executable instructions. The computer executable instructions, when executed by a processor, cause the processor to receive image data generated based on data acquired for a subject during a scan of the subject and a scan protocol. The scan protocol includes a mapping to a file transfer profile. The file transfer profile includes a file transfer location to a host. The scan protocol does not include the file transfer location to the host. The computer executable instructions, when executed by the processor, cause the processor to transfer the image data to the host based on the file transfer location in the file transfer profile.

Those skilled in the art will recognize still other aspects of the present application upon reading and understanding the attached description.

### BRIEF DESCRIPTION OF THE DRAWINGS

The application is illustrated by way of example and not limited by the figures of the accompanying drawings in which like references indicate similar elements.

FIG. 1 schematically illustrates a non-limiting example of an imaging system configured for Computed Tomography (CT) imaging, in accordance with an embodiment(s) herein.

FIG. 2 schematically illustrates an example file transfer profile with only one or more hosts, in accordance with an embodiment(s) herein.

FIG. 3 schematically illustrates another example file transfer profile with one or more hosts and one or more file transfer profiles, in accordance with an embodiment(s) herein.

FIG. 4 schematically illustrates yet another example file transfer profile with only one or more file transfer profiles, in accordance with an embodiment(s) herein.

FIG. 5 schematically illustrates still another example file transfer profile with hosts, file transfer profiles, and transfer priorities for the hosts and the file transfer profiles, in accordance with an embodiment(s) herein.

FIG. 6 schematically illustrates still another example file transfer profile with hosts, file transfer profiles, transfer priorities for the hosts and the file transfer profiles, and types of data to transfer, in FIG. 6 schematically illustrates a set of file transfer protocols, in accordance with an embodiment(s) herein.

FIG. 7 diagrammatically illustrates an example profile manager Graphical User Interface (GUI), in accordance with an embodiment(s) herein.

FIG. 8 diagrammatically illustrates a portion of an example transfer options GUI where no file transfer profile or host is selected, in accordance with an embodiment(s) herein.

FIG. 9 diagrammatically illustrates the portion of the example transfer options GUI where at least a host is selected, in accordance with an embodiment(s) herein.

FIG. 10 diagrammatically illustrates a file transfer profile after a host is added, in accordance with an embodiment(s) herein.

FIG. 11 diagrammatically illustrates the portion of the example transfer options GUI that further includes priorities for the file transfer profiles and/or hosts, in accordance with an embodiment(s) herein.

FIG. 12 diagrammatically illustrates the portion of the example transfer options GUI that includes priorities for the file transfer profiles and/or hosts and options for the type of data to transfer, in accordance with an embodiment(s) herein.

FIG. 13 diagrammatically illustrates a portion of an example system preferences GUI for enabling and disabling file transfer profiles with unpopulated fields, in accordance with an embodiment(s) herein.

FIG. 14 diagrammatically illustrates the portion of the example system preferences GUI for enabling and disabling file transfer profiles with populated fields, in accordance with an embodiment(s) herein.

FIG. 15 diagrammatically illustrates a portion of an example scan-time GUI with a reconstruction transfer option automatically populated with a file transfer profile and a transfer priority where the file transfer profile and the transfer priority cannot be changed within the GUI, in accordance with an embodiment(s) herein.

FIG. 16 diagrammatically illustrates the portion of the example scan-time GUI with the reconstruction transfer option automatically populated with the file transfer profile and the transfer priority where the file transfer profile and/or the transfer priority can be changed within the GUI, in accordance with an embodiment(s) herein.

FIG. 17 diagrammatically illustrates a portion of an example scan-time GUI with a report transfer option automatically populated with a file transfer profile and a transfer priority where the file transfer profile and the transfer priority cannot be changed within the GUI and cannot be changed within the GUI, in accordance with an embodiment(s) herein.

FIG. 18 diagrammatically illustrates the portion of the example scan-time GUI with the report transfer option automatically populated with the file transfer profile and the transfer priority where the file transfer profile and/or the transfer priority can be changed within the GUI, in accordance with an embodiment(s) herein.

FIG. 19 depicts a non-limiting example of a flow chart for a computer-implemented method for employing file transfer profiles in one or more scan protocols to identify file transfer locations for reconstructions in the one or more scan protocols, in accordance with an embodiment(s) herein.

FIG. 20 depicts a non-limiting example of a flow chart for a computer-implemented method for automatically updating network addresses for one or more reconstructions of one or more scan protocols through one or more file transfer profiles included in the one or more reconstructions, in accordance with an embodiment(s) herein.

FIG. 21 depicts a non-limiting example of a flow chart for a computer-implemented method for enabling and/or disabling use of one or more file transfer profiles in one or more reconstructions of one or more scan protocols, in accordance with an embodiment(s) herein.

### DETAILED DESCRIPTION

Embodiments of the present disclosure will now be described, by way of example, with reference to the figures, in which a system, a method and/or a set of instructions on a computer readable medium provide efficient transfer of medical data (e.g., one or more 2-D image slices, a 3-D volume, reports, etc.) from an imaging system to a host based on a file transfer profile mapped to in a scan protocol. In one instance, a file transfer profile includes one or more file transfer locations (e.g., network addresses) to one or more hosts and/or one or more other file transfer profiles that include such file transfer locations. Each scan protocol includes a mapping to one or more file transfer profiles for each reconstruction. As such, each reconstruction is mapped to the one or more file transfer locations of the one or more file transfer profiles. In some instances, the file transfer profiles further include transfer priority levels, transfer data type options, etc. In some instances, automatic selection of a file transfer profile in scan protocols can be enabled/disabled.

With a configuration that does not include or use the file transfer profiles described herein and scan protocols instead include network addresses for each reconstruction, when a file transfer location changes (e.g., a new network address is added, a host is removed, a network address for a host changes, the network changes, etc.), each protocol on each imaging system that is affected by the change needs to be manually updated by authorized personnel to reflect the change, which can be a time consuming task that if not performed could result in medical data not being transferred at all or not being transferred to a correct host. The approach herein mitigates having to have authorized personnel update each affected reconstruction in affected scan protocols on each affected imaging system as each of the affected scan protocols map file transfer profiles, and updating a particular file transfer profile automatically updates each affected reconstruction of each affected protocol with the updated file transfer location.

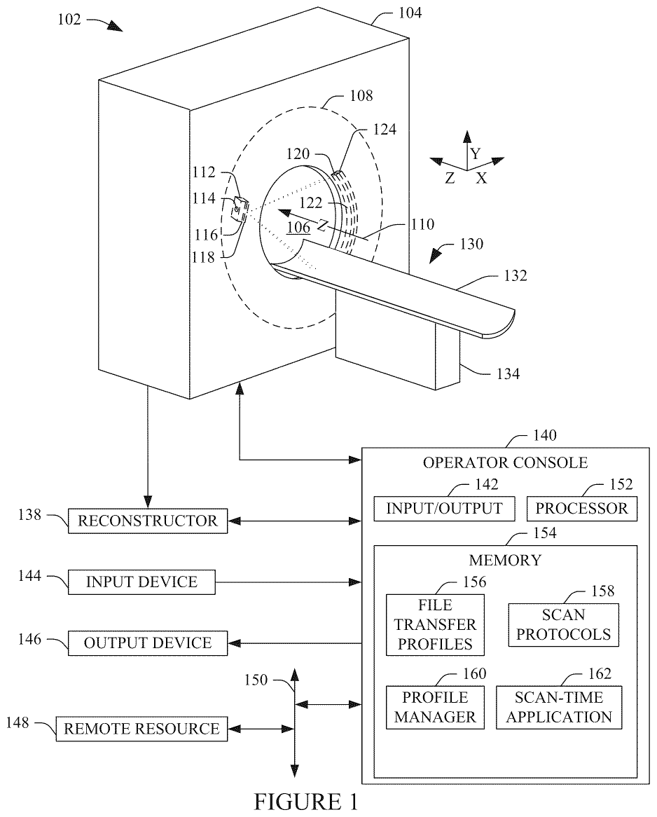

Initially referring to FIG. 1, a non-limiting example of an imaging system 102 is schematically illustrated. In this example, the imaging system 102 is configured for Computed Tomography (CT) imaging. In another instance, the imaging system 102 is additionally or alternatively configured to include another imaging modality such as a Magnetic Resonance (MR) imaging system, an X-ray imaging system, a Positron Emission Tomography (PET) imaging system, a Single Photon Emission Computed Tomography (SPECT) imaging system, and/or other imaging system. For clarity and sake of brevity, the below discussion describes the imaging system 102 configured for CT imaging.

The imaging system 102 includes a gantry 104 with a bore 106. In some instances, the gantry 104 is configured to tilt. The imaging system 102 further includes a rotating frame 108. The rotating frame 108 is rotatably supported in the gantry 104, e.g., via a bearing (e.g., a slip ring) or the like, and is configured to rotate around the bore 106 about a rotational or Z-axis 110, which extends through a center of rotation (e.g., a center of the bore 106, i.e., an isocenter). A gantry controller (not visible) is configured to control rotation of the rotating frame 108 and, if configured to tilt, tilting of the gantry 104.

An X-ray source assembly 112 is supported by the rotating frame 108 and rotates in coordination with the rotating frame 108. The X-ray source assembly 112 includes an X-ray source 114 such as an X-ray tube. The X-ray source 114 is configured to emit X-ray radiation having an energy at least in the X-ray diagnostic range (e.g., 20 keV to 150 keV). The X-ray assembly 112 may further include or is coupled to a filter 116 that characterizes an X-ray radiation dose profile and/or a collimator 118 that shapes the X-ray radiation to form a generally (fan, wedge, cone, etc.) shaped beam that traverses the bore 106. An X-ray controller (not visible) is configured to control components of the X-ray source assembly 112 such as X-ray radiation emission of the X-ray source 114, the collimator 118, etc.

A detector array 120 includes a one-dimensional (1-D) or two-dimensional (2-D) array of rows of X-ray radiation sensitive detector elements 122 and is supported by the rotating frame 108 along an arc opposite the X-ray source 114, across the bore 106. Each of the detector elements 122 is in electrical communication with a Data Acquisition System (DAS) 124. The detector elements 122 include an indirect conversion detector such as a scintillator/photodiode detector and/or a direct conversion detector such as a Cadmium Telluride (CdTe), a Cadmium Zinc Telluride (CZT), etc. detector. A DAS controller (not visible) controls the X-ray radiation sensitive detector array 120.

A table 130 includes a cradle 132 moveably coupled to a frame/base 134. In one instance, the cradle 132 is slidably coupled to the frame/base 134 via a bearing or the like, and a drive system (not visible) including a motor, a lead screw, and a nut (or other drive system) translates the cradle 132 along the frame/base 134 into and out of the bore 106 for horizontal motion, and the frame/base 134 includes a drive system (not visible) including a mechanism for vertical or diagonal motion. The cradle 132 is configured to support a subject in the bore 106 for loading, scanning, and/or unloading. A table controller (not visible) controls the drive system.

For a helical scan, the rotating frame 108 rotates in coordination with the tabletop 132 moving along the Z-axis 110, and active X-ray detector elements 122 of the X-ray radiation sensitive detector array 120 detect X-ray radiation over consecutive arc segments (integration periods) each revolution and generate respective signals. For an axial (step and shoot) scan, the cradle 132 is positioned at a static position for each integration period and moves between integration periods. For each arc segment, the DAS 124 processes each signal and generates projection data.

A reconstructor 138 reconstructs the projection data and generates volumetric (3-D) image data for a helical scan and/or individual axial (2-D) image for an axial step and shoot scan (which can be used in combination to generate volumetric image data). The volumetric image data and/or 2-D slices thereof, and/or the individual axial images can be visually presented, filmed, etc. Examples of suitable reconstruction algorithms include filtered back projection (FBP), advanced statistical iterative reconstruction (ASIR), conjugate gradient (CG), maximum likelihood expectation maximization (MLEM), model-based iterative reconstruction (MBIR), and/or other reconstruction algorithm.

A computing system 140 serves as an “OPERATOR CONSOLE” of the imaging system 102. The computing system 140 may include a computer, a workstation, server, etc. The computing system 140 includes input/output (I/O) 142. An input device 144 includes a keyboard, mouse, touchscreen, microphone, etc. The input device 144 is in electrical communication with the computing system 140 through the I/O 142 and/or otherwise. An output device 146 includes a human readable device such as a display monitor or the like. The output device 146 is in electrical communication with the computing system 140 through the I/O 142 and/or otherwise.

A remote resource 148 includes one or more of a server, a workstation, a Radiology Information System (RIS), a Hospital Information System (HIS), an Electronic Medical Record (EMR), a PACS, a PACS configured with image viewing and/or manipulating software, cloud resources with shared remote data storage and/or computing power including resources distributed over data centers, etc. The computing system 140 and the remote resource 148 are in communication with a network 150 that includes wired and/or wireless technologies. Communication over the network 150 can be through Digital Imaging and Communications in Medicine (DICOM), Health Level Seven (HL7), etc. formats and protocols.

In one instance, the network 150 is configured for communicating medical data (e.g., one or more 2-D slices, a 3-D volume, reports, DICOM images, screen captures, etc.) amongst the computing system 140 and one or more components of the remote resource 148. An example of a report includes dose related reports such as a Dose Structured Report (SR), a Dose Secondary Capture (SC) report, a Contrast Report, etc. Such reports include information about the radiation dose a patient receives during a CT scan, images not directly acquired from the CT scanner, information about the use of contrast agents during the CT scan, etc. Other reports are contemplated herein.

The computing system 140 further includes at least one processor 152 such as a microprocessor (μP), a central processing unit (CPU), graphics processing unit (GPU), etc., and a computer readable medium 154 (“MEMORY”), which includes non-transitory medium and excludes transitory medium (signals, carrier waves, and the like). In the illustrated example, the computer readable medium/memory 154 at least includes file transfer profiles 156, scan protocols 158, a profile manager 160, and a scan-time application 162. In another instance, at least one of the transfer file profiles 156, the scan protocols 158 and the profile manager 160 resides outside of the operator console, such as with one or more systems of the remote resource 148.

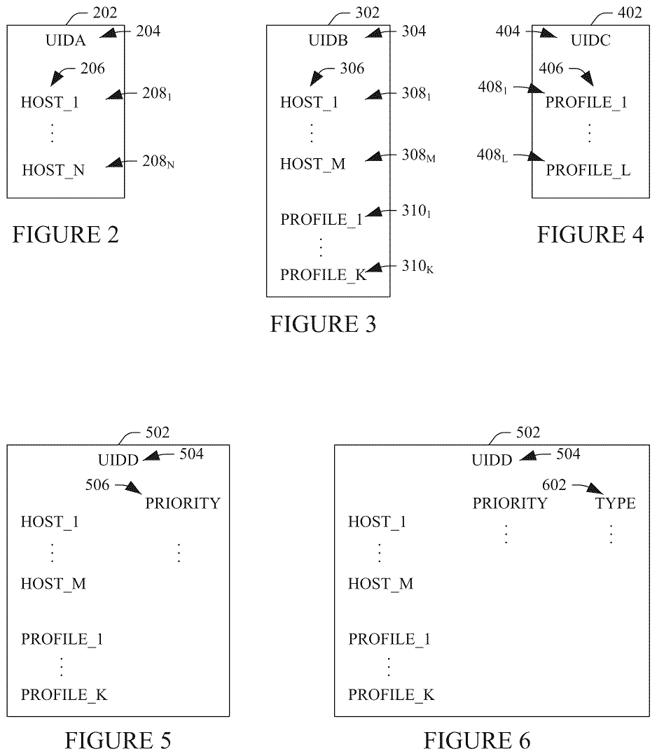

An example file transfer profile of the file transfer profiles 156 at least includes a unique identifier (UID) that uniquely identifies the file transfer profile (e.g., from other file transfer profiles of the file transfer profiles 156) and one or more file transfer locations (e.g., one or more network addresses) to one or more hosts (e.g., at least one entity accessible via the network 150 such as an entity of the remote resource 148 and/or otherwise). Turning to FIG. 2, an example file transfer profile 202 is diagrammatically illustrated.

The file transfer profile 202 includes a field 204 for a unique identifier (UID) and a region 206 with one or more fields for network addresses (and/or aliases to network addresses). As utilized herein, the term “HOST” in the figures represents the network address or the alias to the network address. For explanatory purposes, the field 204 is populated with a unique identifier “UIDA,” and the region 206 is populated with N file transfer locations, including a first file transfer location (“HOST_1”) 208₁, . . . , and an Nth file transfer location (“HOST_N”) 208_{N}, wherein N is a positive integer equal to or greater than one.

A scan protocol with a reconstruction that maps the file transfer profile 202 (“UIDA”) as a transfer location will automatically transfer medical data for the reconstruction using the file transfer locations in the “UIDA” file transfer profile 202. In this example, the data would be transferred to the network addresses corresponding to “HOST_1,” . . . , and “HOST_N.” For instance, where “HOST_1” identifies the network address w.x.y.z and a reconstruction in a protocol maps the file transfer profile “UIDA” as a file transfer location, the imaging system 102 will automatically transfer the medical data to the host at the network address w.x.y.z. This would apply for all of the file transfer locations.

FIG. 3 diagrammatically illustrates an example file transfer profile 302, which is a variation of the file transfer profile 202 depicted in FIG. 2. The file transfer profile 302 similarly includes a field 304 for a unique identifier and a region 306 with one or more fields for network addresses and/or aliases to network addresses. In this example, the region 306 further includes one or more fields for identifying one or more other file transfer profiles. In this example, for explanatory purposes, the field 304 is populated with a unique identifier “UIDB.”

The region 306 is populated with M transfer locations, including a first transfer location (“HOST_1”) 308₁, . . . , and an Mth transfer location (“HOST_M”) 308_{M}, wherein M is a positive integer equal to or greater than one. The region 306 is further populated with L file transfer profiles, including a first file transfer profile (“PROFILE_1”) 310₁, . . . , and a Kth transfer profile (“PROFILE_K”) 310_{K}, wherein K is a positive integer equal to or greater than one. As utilized herein, the term “PROFILE” in the figures represents a file transfer profile such as “UIDA,” etc.

A scan protocol with a reconstruction that maps the file transfer profile 302 (“UIDB”) as a file transfer location will automatically transfer medical data using the file transfer locations identified in the “UIDB” file transfer profile 302. In this example, the medical data would be transferred using the network addresses corresponding to “HOST_1,” . . . , and “HOST_M.” For instance, where “HOST_M” identifies the network address w.y.x.z and a reconstruction in a scan protocol maps the file transfer profile “UIDB” as a file transfer location, the imaging system 102 will automatically transfer medical data to the host at the network address w.x.y.z1. This would apply for all of the file transfer locations 308.

Where the Kth file transfer profile 310_{K} is the file transfer profile 202 (FIG. 2), i.e., “PROFILE_K=UIDA,” the imaging system 102 will also automatically transfer the medical data to the file transfer locations in the “UIDA” file transfer profile 202. As such, the image data would also be transferred using the network addresses corresponding to “HOST_1,” . . . , and “HOST_N.” For instance, where “HOST_1” of the “UIDA” file transfer profile 202 identifies the network address w.x.y.z2, the imaging system 102 will also automatically transfer medical data to the host at the network address w.x.y.z2. This would apply for all of the file transfer profiles 308.

FIG. 4 diagrammatically illustrates an example file transfer profile 402. The file transfer profile 402 includes a field 404 for a unique identifier and a region 406 with one or more fields for identifying other file transfer profiles. In this example, for explanatory purposes, the field 404 is populated with a unique identifier “UIDC.” The region 406 is populated with L file transfer profiles, including a first file transfer profile (“PROFILE_1”) 408₁, . . . , and an Lth transfer profile (“PROFILE_L”) 408_{L}, wherein L is a positive integer equal to or greater than one.

A protocol with a reconstruction that maps the file transfer profile 402 (“UIDC”) as a file transfer location will automatically transfer data using the transfer locations in the “UIDC” file transfer profile 402. Where the Lth transfer file profile 408_{L} includes the transfer file profile 202 “UIDA” (FIG. 2), the imaging system 102 will automatically transfer the medical data using the file transfer locations in the “UIDA” file transfer profile 202, as described in connection with FIG. 2.

FIG. 5 diagrammatically illustrates an example file transfer profile 502, which is a variation of the file transfer profile 302 depicted in FIG. 3. The file transfer profile 502 includes a field 504 for a unique identifier, the region 306 (FIG. 3), and a transfer priority level 506 for at least one of the hosts 308₁, . . . , 308_{M} and/or file transfer profiles 310₁, . . . , 310_{K}. The at least one transfer priority level 506 determines a file transfer order. For example, medical data for network addresses with a higher priority level will be transferred before medical data for network addresses with a lower priority level.

Where multiple reconstructions in a scan protocol include network addresses with a same transfer priority level and the medical data of the multiple reconstructions can be concurrently transferred, the imaging system 102 concurrently transfers the medical data for the multiple reconstructions. Where the image data cannot be concurrently transferred, imaging system 102 concurrently transfers the medical data based on a predetermined approach such as a first in, first out (FIFO) approach in which the data that was ready first is transferred first. Other approaches are contemplated herein.

In one instance, the transfer priority level 506 further includes other information that in combination with the priority level determines a file transfer order. For example, a higher priority level (e.g., for a trauma reconstruction) may also indicate that the data should be transferred within a predetermined time frame, while a lower priority level may also indicate that the data can be transferred when network traffic is low, there are no further examinations scheduled for the imaging system 102 (FIG. 1), during hours when the imaging entity is closed, etc.

FIG. 6 diagrammatically illustrates a variation of the file transfer profile 502 (FIG. 5) that further includes a “type” field 602. In one instance, the type field 602 allows a user to select a type of medical data to transfer for each host and/or file transfer profile. In one instance, the data includes two types, images (e.g., one or more 2-D slices, a 3-D volume, etc.) and reports, and the options include reports only or images and reports. Other types and/or options are also contemplated herein.

Returning to FIG. 1, an example scan protocol of the scan protocols 158 includes, as briefly discussed herein, at least parameters utilized for scanning (e.g., parameters for acquiring projection data and reconstructing image data from the acquired projection data) and parameters utilized for transferring the image data to one or more hosts.

For example, a head CT protocol might include a non-contrast series. In another example, a body CT protocol might include multiple series to cover different anatomical regions. In another example, an abdomen and pelvis CT protocol might include a non-contrast series and one or more contrast-enhanced series to capture different contrast phases (e.g., arterial, portal, delayed, etc.). In another example, a trauma CT protocol might include different series to capture multiple body regions such as the head, neck, chest, abdomen, pelvis, etc. Other protocols are also contemplated herein.

Each of the different series includes one or more reconstructions. For example, with the trauma CT protocol, the head series may include one or more reconstructions, the neck series may include one or more reconstructions, the chest series may include one or more reconstructions, the abdomen series may include one or more reconstructions, the pelvis series may include one or more reconstructions, etc. Each of the one or more reconstructions maps one or more of the file transfer profiles 156. As such, each of the one or more reconstructions includes the one or more of the file transfer locations in the one or more of the file transfer profiles 156.

With the scan protocols 158 including the one or more of the file transfer profiles 156, none of the scan protocols 158 would need to be updated were a file transfer location added, a file transfer location removed and/or a file transfer location changed. By way of non-limiting example, if the head CT scan protocol, the body CT scan protocol, the abdomen and pelvis CT scan protocol, the trauma CT scan protocol, etc. all included a file transfer profile with a same file transfer location to a particular host and the file transfer location for the host were modified, only the file transfer profile would need to be updated, and not the individual head, body, abdomen and pelvis, trauma, etc. scan protocols.

As discussed herein, in a configuration that does not include or use the file transfer profiles 156 and the scan protocols themselves include file transfer locations under each reconstruction, each of the scan protocols 158 that included a modified file transfer location would need to be manually updated by authorized personnel, including each reconstruction in a scan protocol that included the file transfer location that was modified. In general, this would require authorized personnel to manually update each of the reconstructions in each of the scan protocols affected by the file transfer location modification each time there is a modification to file transfer locations.

The profile manager 160 is configured to allow a user to manage the file transfer profiles 156, including create, remove, change, etc. file transfer profiles of the file transfer profiles 156. This at least includes managing which file transfer locations and/or other file transfer profiles are included in a file transfer profile of the file transfer profiles 156. In one instance, this further includes identifying a transfer priority level for one or more network addresses in the file transfer profile and/or identifying the type of data (e.g., image data and/or non-image data) that will be transferred to each transfer location.

The scan-time application 162 allows the clinician to select and open a scan session and scan protocol of the file transfer profiles 156 for a scan of a subject and enter parameters into the scan protocol. As discussed herein, this includes entering parameters such as X-ray tube voltage, X-ray tube current, slice thickness, scan range, rotation time, etc., subject positioning, one or more image acquisitions (e.g., pre-scan/scout, helical, axial, etc.), one or more reconstructions, etc. The open scan protocol, which includes one or more of the file transfer profiles 156, will automatically include the file transfer location from the one or more of the file transfer profiles 156 mapped to in the scan protocol.

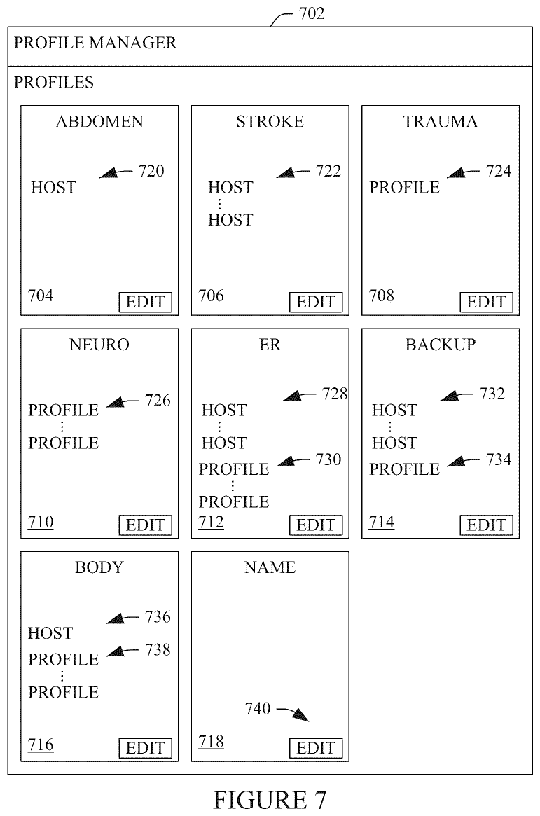

As described herein, each of the file transfer profiles 156, for each reconstruction therein, at least includes one or more file transfer locations and/or another file transfer profile that includes one or more file transfer locations. In one instance, management of the file transfer profiles 156 is provided through graphical user interface (GUI). FIG. 7 diagrammatically illustrates an example of a graphical user interface (GUI) 702 configured to manage file transfer profiles of the file transfer profiles 156. In this example, the GUI 702 already includes a plurality of file transfer profiles.

As discussed herein, each file transfer profile includes a UID. In this example, there is an abdomen (“UID”=“ABDOMEN”) file transfer profile 704, a stroke (“UID”=“STROKE”) file transfer profile 706, a trauma (“UID”=“TRAUMA”) file transfer profile 708, a neuro (“UID”=“NEURO”) file transfer profile 710, an emergency room (“UID”=“ER”) file transfer profile 712, a backup (“UID”=“BACKUP”) file transfer profile 714, and a body (“UID”=“BODY”) file transfer profile 716. A template (“UID”=“NAME”) file transfer profile 718 provides a template for adding additional file transfer profiles. In this example, a user would rename the file transfer profile 718 and add a host and/or profile via an edit control.

In this example, the abdomen file transfer profile 704 includes a single host 720, the stroke file transfer profile 706 includes multiple hosts 722, the trauma file transfer profile 708 includes a single profile 724, the neuro file transfer profile 710 includes multiple hosts 726, the ER file transfer profile 712 includes multiple hosts 728 and multiple profiles 730, the backup file transfer profile 714 includes multiple hosts 732 and a single profile 734, and the body file transfer profile 716 includes a single host 736 and multiple profiles 738. In other examples, one or more of the transfer profiles 720-738 include different hosts and/or file transfer profiles.

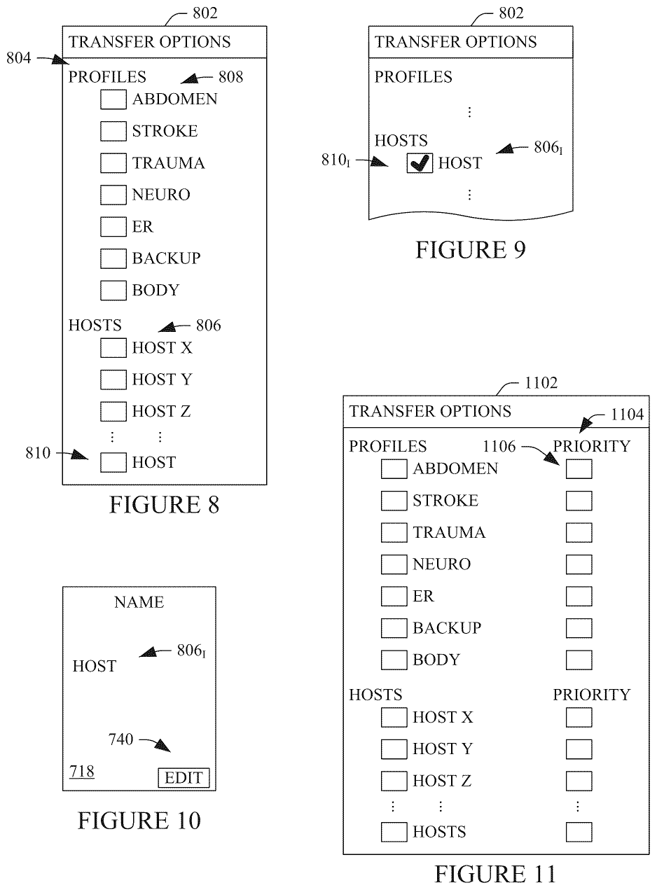

Each of the file transfer profiles 720-738 includes an edit control 740 for editing profile, e.g., for adding, removing and/or changing a host or file transfer profile. For example, in one instance, invoking the edit control 740, e.g., of the template file transfer profile 718, provides options for hosts and/or file transfer profiles. FIG. 8 diagrammatically illustrates an example transfer options GUI 802, configured to manage hosts and/or file transfer profiles options. In this example, the GUI 802 includes the hosts 806 and the file transfer profiles 808. In this example, each of the profiles 806 and each of the hosts 806 includes a checkbox control 810.

A user can toggle a state of the checkbox control 810 between unchecked in which the checkbox control 810 is empty (as shown in FIG. 8) and checked in which the checkbox control 810 includes a “check” (as shown in FIG. 9). A user can toggle between unchecked and checked states, e.g., via clicking with a mouse pointer, a stylus, an anatomical digit (e.g., a finger), etc. Checking a checkbox adds the profile or the host to the template file transfer profile 718.

FIG. 9 diagrammatically illustrates a sub-portion of the transfer options GUI 802 depicting a checked checkbox 810_{I} for a host 806_{I}. FIG. 10 diagrammatically illustrates the template file transfer profile 718 (FIG. 7) updated to include the host 806_{I}. Unchecking the checked checkbox 810_{I} would remove the host 806_{I} from the template file transfer profile 718. Checking a checkbox (FIG. 8) for another host and/or a file transfer profile would additionally add the other host and/or the file transfer profile corresponding to the checked checkbox. Other approaches for adding and/or removing hosts and/or file transfer profiles are contemplated herein.

FIG. 11 diagrammatically illustrates an example transfer options GUI 1102, which is a variation of the example transfer options GUI 802 (FIG. 8). In this variation, the transfer options GUI 1102 further includes priority fields 1104, e.g., as discussed in connection with FIG. 5 herein. Again, data will be transferred to a host with higher priority before data is transferred to a host with lower priority. In this example, each of the hosts 804 includes a priority control 1106. Examples of priority controls includes a button that toggles between multiple different priority levels, a drop-down menu with different selectable priority levels, a pop-up menu with different selectable priority levels, etc. In another instance, at least one of the hosts and/or profiles does not include a priority field, e.g., a host and/or profile that includes a static priority field, e.g., for a trauma protocol.

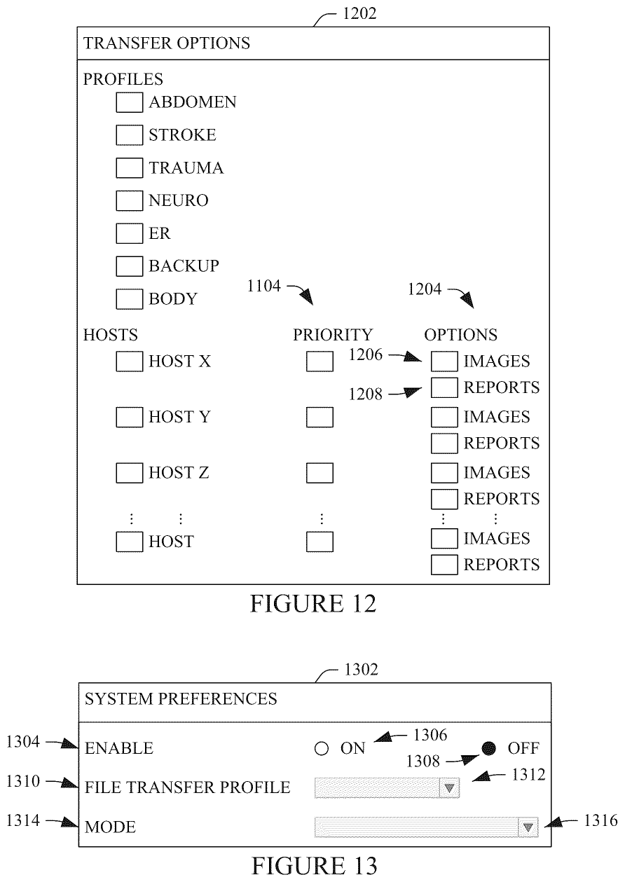

FIG. 12 diagrammatically illustrates another example transfer options GUI 1202, which is a variation of the example transfer options GUI 1102 (FIG. 11). In this variation, the transfer options GUI 1202 not only includes the priority fields 1104, but further includes an options field 1204, e.g., as discussed in connection with FIG. 6 herein. In this example, each of the hosts includes a set of options checkboxes. A user can toggle a state of a checkbox between unchecked in which the checkbox control is empty and checked in which the checkbox includes a check. A user can toggle between unchecked and checked states, e.g., via clicking with a mouse pointer, a stylus, an anatomical digit, etc.

In this example, the set of options checkboxes 1204 includes an option checkbox 1206 for images (e.g., one or more 2-D image slices, a 3-D volume, etc.) and an options checkbox 1208 for reports (e.g., SR, SC, contrast, etc. reports). Checking the options checkbox 1206 indicates images will be automatically transferred to the host, checking the options checkbox 1208 indicates reports will be automatically transferred to the host, and checking both options checkboxes 1206 and 1208 indicates that both images and reports will be automatically transferred to the host. Although FIG. 12 only shows the options 1204 for the hosts, in another instance, the options 1204 are only for the profiles, or are for both the hosts and the profiles.

Again, a scan protocol, for each reconstruction, will include one or more of the file transfer profiles 156 (FIGS. 1 and 6). The operator console 140 is automatically configured to send medical data to the network addresses identified in the one or more of the file transfer profiles 156. As such, where a file transfer profile of the file transfer profiles 156 is updated to add, remove and/or change a network address, all protocols with reconstructions that map the file transfer profile will automatically include the addition and/or change, and/or no longer include any removed network address.

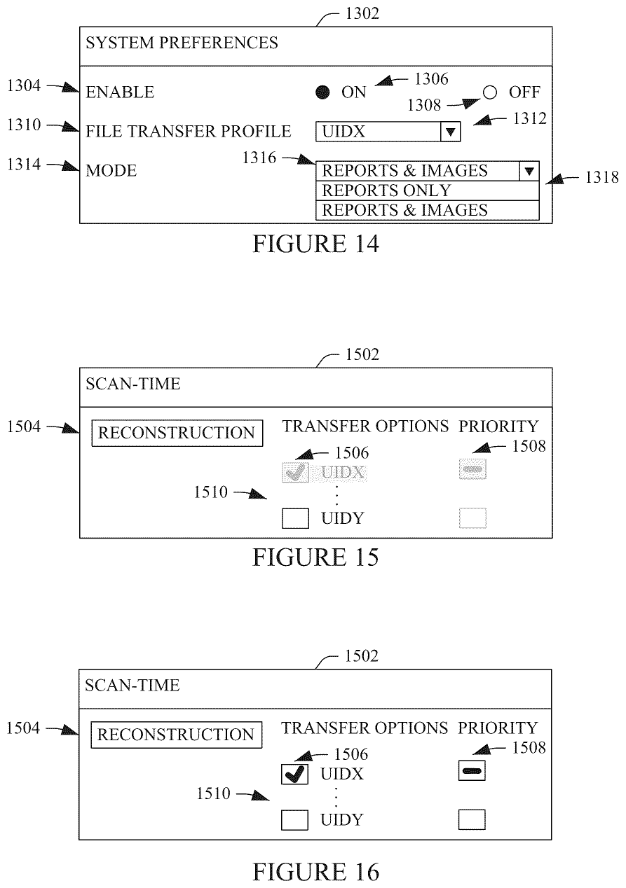

In the above example, each scan protocol of the scan protocols 158, for each reconstruction, includes one or more of the file transfer profiles 156, which automatically includes the network addresses of the one or more of the file transfer profiles 156 for each reconstruction. FIGS. 13 and 14 graphically illustrates a portion of an example system preferences GUI 1302 that allows an imaging entity to enable or disable automatic use of a file transfer profile for scan protocols.

The system preferences GUI 1302 includes a field 1304 with a graphical control that includes user selectable control widgets. In this example, the user selectable control widgets include an enable widget (“ON”) 1306 and a disable widget (“OFF”) 1308. Only one of the widgets 1306 or 1308 can be selected at any point in time, and changing which of the widgets 1306 or 1308 is selected automatically unselects the other widget. The illustrated widgets 1306 or 1308 include radio buttons. In another example, the widgets include checkboxes, a toggle switch, and/or other graphical control.

The system preferences GUI 1302 further includes a field 1310 with a graphical control widget with user selectable options of pre-existing file transfer profile. In this example, the user selectable options are included in a widget 1312 that includes a list of available file transfer profiles. The illustrated widget 1312 include a drop-down list. In another example, the widget 1312 includes a list box, a slider, and/or other graphical control.

The system preferences GUI 1302 further includes a field 1314 with a graphical control widget with user selectable pre-existing mode options. In this example, the user selectable pre-existing mode options are included in a widget 1316 that includes a list of available modes, which indicate the type of data to transfer (e.g., image data and reports). The illustrated widget 1316 includes a drop-down list. In another example, the widgets 1316 includes a list box, a slider, and/or other graphical control.

As shown in FIG. 13, in one instance, when the disable widget 1308 is selected (as indicated by the filled (black) widget 1308), the widget 1312 and the widget 1316 are inactive. As shown in FIG. 14, in one instance, when the enable widget 1306 is selected (as indicated by the filled (black) widget 1306), both the widget 1312 and the widget 1316 are active, and a user can select a file transfer protocol and a mode. In FIG. 14, a file transfer profile has already been selected from the drop-down list widget 1312, and the mode options list widget 1316 presents a list of mode options 1318. The mode options list widget 1316 includes a least two mode options.

FIG. 15 diagrammatically illustrates a portion of an example scan-time GUI 1502 that shows reconstruction 1504 file transfer profiles and transfer priority level. FIG. 15 corresponds to FIG. 14 in which a user selected a file transfer profile with the widget 1312 and a mode with the widget 1316. In FIG. 15, a checkbox 1506 for the reconstruction file transfer profile is automatically checked and a priority level field 1508 is automatically populated. In this example, the checkbox 1506 and the priority level field 1508 are inactive and a user cannot change them in the GUI 1502. In this example, the transfer options include one or more other selectable other file transfer options 1510. Where the option “REPORTS ONLY” is selected in the preferences GUI 1302 in FIG. 14, the checkbox 1506 would not be automatically checked.

FIG. 16 diagrammatically illustrates a portion of the example scan-time GUI 1502 that shows reconstruction 1504 file transfer profiles and transfer priority level. FIG. 16 corresponds to FIG. 14 in which a user selected a file transfer profile with the widget 1312 and a mode with the widget 1316. In FIG. 16, again the checkbox 1506 for the file transfer profile is automatically checked and the priority level field 1508 is automatically populated. However, in this example, the checkbox 1506 and the priority level field 1508 are active and the user can change them. In this example, the transfer options include one or more other selectable other file transfer options 1510. Where the option “REPORTS ONLY” is selected in the preferences GUI 1302 in FIG. 14, the checkbox 1506 would not be automatically checked.

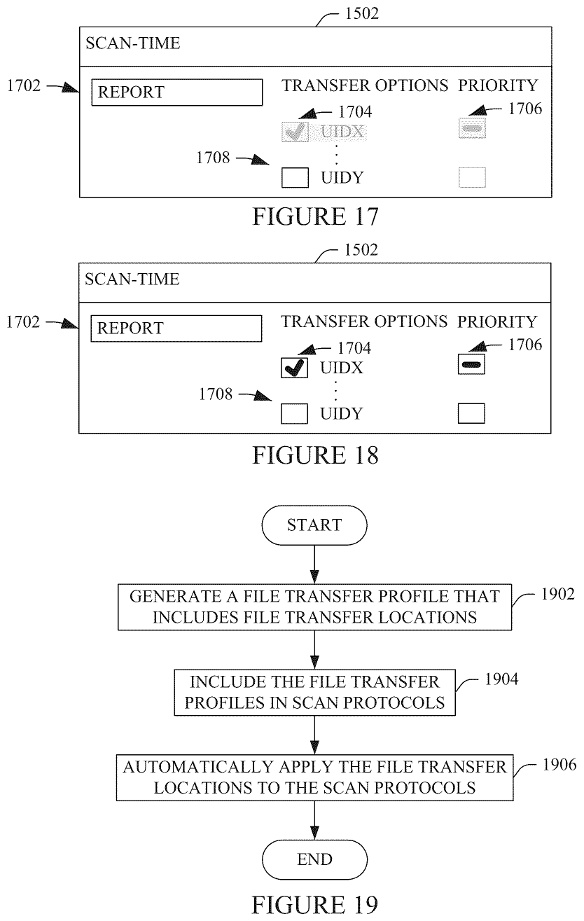

FIG. 17 diagrammatically illustrates a portion of an example scan-time GUI 1502 that shows report 1702 file transfer profiles and transfer priority level. FIG. 17 corresponds to FIG. 14 in which a user selected a file transfer profile with the widget 1312 and a mode with the widget 1316. In FIG. 17, a checkbox 1704 for the mode file transfer profile is automatically checked and a priority level field 1706 is automatically populated. In this example, the transfer options include one or more other selectable other file transfer options 1708.

FIG. 18 diagrammatically illustrates a portion of an example scan-time GUI 1502 that shows report 1702 file transfer profiles and transfer priority level. FIG. 18 corresponds to FIG. 14 in which a user selected a file transfer profile with the widget 1312 and a mode with the widget 1316. In FIG. 18, again the checkbox 1704 for the file transfer profile is automatically checked and the priority level field 1706 is automatically populated. However, in this example, the checkbox 1704 and the priority level field 1706 are active and the user can change them.

FIG. 19 illustrates a non-limiting example of a flow chart for employing file transfer profiles in one or more scan protocols to identify file transfer locations for reconstructions in the one or more scan protocols, in accordance with an aspect herein. It is to be appreciated that the ordering of the acts in the method is not limiting. As such, other orderings are contemplated herein. In addition, one or more acts may be omitted, and/or one or more additional acts may be included.

At 1902, a file transfer profile that includes one or more file transfer locations is generated, as described herein and/or otherwise. For example, the GUI 702 described in connection with FIG. 7 shows several examples of file transfer profiles, and the GUIs 802, 1102 and 1202 described in connection with FIGS. 8, 9, 11 and 12 show examples for editing file transfer profiles. The file transfer profile 718 described in connection with FIG. 10 shows the addition of host thereto in response to a selection of the host in the GUI 802.

At 1904, one or more file transfer profiles are included in one or more scan protocols, as described herein and/or otherwise. For example, for each reconstruction in each protocol, a protocol maps one or more of the file transfer profiles. As discussed herein, identifying the one or more of the file transfer profiles for one or more reconstructions in the one or more scan protocols mitigates having to add, remove and/or change one or more network addresses for one or more reconstructions of one or more scan protocols.

At 1906, the file transfer locations are automatically applied to the scan protocols, as described herein and/or otherwise. For example, during a scan session, when a user opens a scan protocol, the file transfer locations in the files transfer profiles for the reconstructions of the scan protocol automatically become the file transfer locations for the reconstructions of the scan protocol.

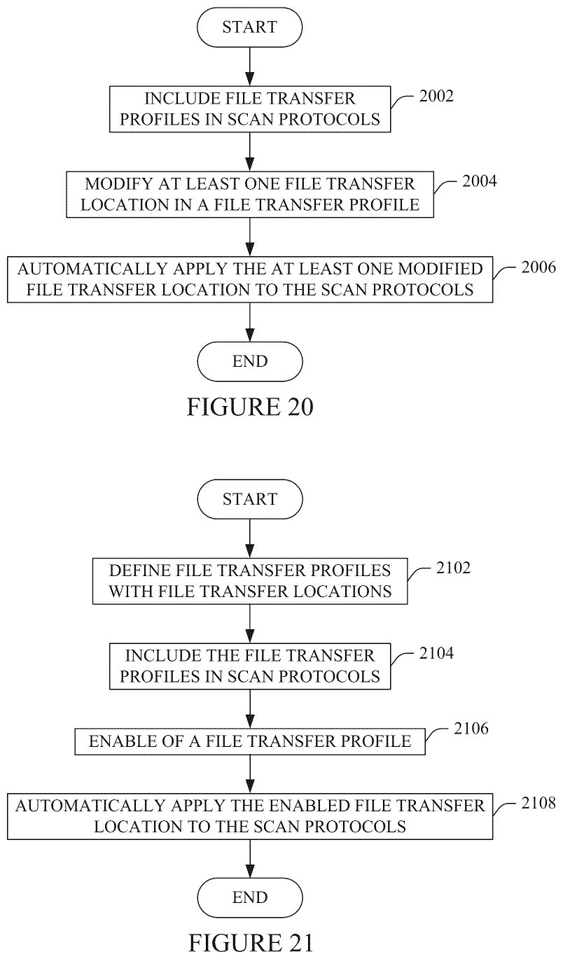

FIG. 20 illustrates another non-limiting example of a flow chart for automatically updating network addresses for one or more reconstructions of one or more scan protocols through one or more file transfer profiles included with the one or more reconstructions, in accordance with an aspect herein. It is to be appreciated that the ordering of the acts in the method is not limiting. As such, other orderings are contemplated herein. In addition, one or more acts may be omitted, and/or one or more additional acts may be included.

At 2002, one or more file transfer profiles are included in one or more scan protocols, as described herein and/or otherwise. Again, for each reconstruction in each protocol, a scan protocol maps one or more of the file transfer profiles, and mapping the one or more of the file transfer profiles for a reconstruction in a scan protocol automatically includes the one or more of the file transfer profiles in the scan protocol.

At 2004, at least one file transfer location in a file transfer profile is modified, as described herein and/or otherwise. For example, adding and/or removing a host is described in connection with the GUI 802 described in FIG. 9 and the file transfer profile 718 described in FIG. 10. In this instance, the user selects a host (and/or other file transfer profile) to add (and/or remove) by toggling a checkbox in the GUI 802 described in FIG. 9, which adds (and/or removes) the host (and/or other file transfer profile) to the file transfer profile 718 described in FIG. 10.

At 2006, the modified file transfer location is automatically applied to the scan protocols, as described herein and/or otherwise. As discussed herein, during a scan session, when a user opens a scan protocol, the file transfer locations in the files transfer profiles for the reconstructions of the scan protocol automatically become the file transfer locations for the reconstructions of the scan protocol, even when a file transfer location of one or more of the files transfer profiles for the reconstructions of the scan protocol has changed since the last time the scan protocol was opened in a scan-session.

FIG. 21 illustrates a non-limiting example of a flow chart for enabling and/or disabling use of one or more file transfer profiles in one or more reconstructions of one or more scan protocols, in accordance with an aspect herein. It is to be appreciated that the ordering of the acts in the method is not limiting. As such, other orderings are contemplated herein. In addition, one or more acts may be omitted, and/or one or more additional acts may be included.

At 2102, one or more file transfer protocols that include file transfer locations are defined, as described herein and/or otherwise. Again, the GUI 702 described in connection with FIG. 7 shows several examples of file transfer profiles, which at least include a UID and one or more hosts and/or one or more file transfer profiles, and the GUIs 802, 1102 and 1202 described in connection with FIGS. 8, 9, 11 and 12 show examples for editing file transfer profiles. The file transfer profile 718 described in connection with FIG. 10 shows the addition of host thereto in response to a selection of the host in the GUI 802.

At 2104, file transfer profiles of the one or more file transfer protocols are included in scan protocols, as described herein and/or otherwise. As discussed herein, for each reconstruction in each protocol, a protocol identifies one or more of the file transfer profiles, and identifying the one or more of the file transfer profiles for one or more reconstructions in the one or more scan protocols automatically includes the one or more of the file transfer profiles in the scan protocol.

At 2106, a file transfer profile is enabled, as described herein and/or otherwise. For example, the GUI 1302 described in connection with FIGS. 13 and 14 show an example in which a user enables or disables use of a file transfer profile through selection of a radio control. When enabled, the user can further identify the file transfer profile and a type of data for automatic transfer. When disabled, in this example, the fields for selecting a file transfer profile and a type of data are not active.

At 2108, the enabled file transfer location is automatically applied to the scan protocols, as described herein and/or otherwise. For example, the GUI 1502 described in connection with FIGS. 15 and 16 show automatic population of the enabled file transfer location for images in a scan-time session, and the GUI 1502 described in connection with FIGS. 17 and 18 show automatic population of the enabled file transfer location for reports in the scan-time session

The above can be implemented by way of computer readable instructions, encoded, or embedded on the computer readable storage medium, which, when executed by a computer processor, cause the processor to carry out the described acts or functions. Additionally, or alternatively, at least one of the computer readable instructions is carried out by a signal, carrier wave or other transitory medium, which is not computer readable storage medium.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include such additional elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”. The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the invention without departing from their scope. While the dimensions and types of materials described herein are intended to define the parameters of the various embodiments of the invention, the embodiments are by no means limiting and are exemplary embodiments. Many other embodiments will be apparent to those of skill in the art upon reviewing the above description.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present disclosure. That is, features of the described embodiments can be combined with any appropriate aspect described above and optional features of any one aspect can be combined with any other appropriate aspects. Similarly, features set forth in dependent claims can be combined with non-mutually exclusive features of other dependent claims, particularly where the dependent claims depend on the same independent claim. Single claim dependencies may have been used as practice in some jurisdictions that require them, but this should not be taken to mean that the features in the dependent claims are mutually exclusive.

What is claimed is:

1. A medical imaging system, comprising:
a data acquisition system for acquiring projection data of a subject during a scan of the subject based on a scan protocol,
wherein the scan protocol includes a reconstruction with a mapping to a file transfer profile, the file transfer profile includes a file transfer location to a host, and the reconstruction does not include the file transfer location;
a reconstructor configured to generate image data based on the acquired data and the scan protocol; and
an operator console with a processor configured to execute instructions that initiate transfer of the image data to the host based on the file transfer location in the file transfer profile mapped to in the reconstruction of the scan protocol.

2. The medical imaging system of claim 1, wherein the file transfer profile includes a transfer priority level for the file transfer location, and the processor transfers the image data to the host based on the transfer priority level.

3. The medical imaging system of claim 1, wherein the file transfer profile includes another file transfer profile that includes the file transfer location, and the processor transfers the image data to the host based on the file transfer location in the other file transfer profile.

4. The medical imaging system of claim 3, wherein the other file transfer profile includes a transfer priority level for the file transfer location, and the processor transfers the image data based on the transfer priority level.

5. The medical imaging system of claim 1, wherein the processor transfers the image data to a different file transfer location based on a change to the file transfer profile without a change to the scan protocol.

6. The medical imaging system of claim 1, wherein the processor transfers the image data to a second host based on a second file transfer location added to the file transfer profile without a change to the scan protocol.

7. The medical imaging system of claim 1, wherein the processor receives a first user input that enables the file transfer profile, and the file transfer profile is automatically selected in the scan protocol each time the scan protocol is utilized.

8. The medical imaging system of claim 7, wherein the processor receives a second user input identifying the file transfer profile.

9. The medical imaging system of claim 8, wherein the processor receives a third user input indicating a type of data to transfer, wherein the type of data includes reports, the image data or the reports and the image data.

10. The medical imaging system of claim 7, wherein the processor receives a second user input that disables the file transfer profile, and the file transfer profile is not automatically selected in the scan protocol.

11. A computer-implemented method, comprising:
receiving image data generated based on data acquired for a subject during a scan of the subject and a scan protocol;
wherein the scan protocol includes a mapping to a file transfer profile, the file transfer profile includes a file transfer location to a host, and the scan protocol does not include the file transfer location to the host; and
transferring the image data to the host based on the file transfer location in the file transfer profile.

12. The computer-implemented method of claim 11, wherein the file transfer profile includes a transfer priority level for the file transfer location, and further comprising transferring the image data to the host based on the transfer priority level.

13. The computer-implemented method of claim 11, further comprising automatically transferring the image data to a different file transfer location based on a change to the file transfer profile.

14. The computer-implemented method of claim 11, further comprising:
automatically selecting the file transfer profile in the scan protocol each time the scan protocol is utilized in response enabling automatic data transfer.

15. The computer-implemented method of claim 14, further comprising:
receiving a second user input indicating a type of data to transfer, wherein the type of data includes reports, the image data or the reports and the image data.

16. A computer readable medium encoded with computer executable instructions, which, when executed by a processor, causes the processor to:
receive image data generated based on data acquired for a subject during a scan of the subject and a scan protocol;
wherein the scan protocol includes a mapping to a file transfer profile, the file transfer profile includes a file transfer location to a host, and the scan protocol does not include the file transfer location to the host; and
transfer the image data to the host based on the file transfer location in the file transfer profile.

17. The computer readable medium of claim 16, wherein the file transfer profile includes a transfer priority level for the file transfer location, and the computer executable instructions further cause the processor to: transfer the image data to the host based on the transfer priority level.

18. The computer readable medium of claim 16, wherein the computer executable instructions further cause the processor to: automatically transfer the image data to a different file transfer location based on a change to the file transfer profile.

19. The computer readable medium of claim 16, wherein the computer executable instructions further cause the processor to:
automatically select the file transfer profile in the scan protocol each time the scan protocol is utilized in response to a first user input enabling automatic data transfer.

20. The computer readable medium of claim 19, wherein the computer executable instructions further cause the processor to:
receive a second user input indicating a type of data to transfer, wherein the type of data includes reports, the image data or the reports and the image data.
